(19) United States
(12) Patent Application Publication
(10) Pub. No.: US 2024/0395420 A1
(43) Pub. Date: Nov. 28, 2024

(54) SYSTEMS AND METHODS FOR DYNAMIC EPIDEMIC MODELING AND ABATEMENT

(71) Applicant: STAGE ANALYTICS, Suwanee, GA (US)

(72) Inventors: Caleb ALEXANDER, Baltimore, MD (US); William PADULA, Rancho Palos Verdes, CA (US); Ben COHEN, Thousand Oaks, CA (US); Rob RAYL, Duluth, GA (US); Natalie REID, Severna Park, MD (US); Rafi ZABIAN, Roswell, GA (US); Rebecca CARRARA, Providence, RI (US); Fran CHINGCUANCO, Baltimore, MD (US); Cullen CONWAY, Portlan, OR (US); Elena FERNANDEZ, New York, NY (US)

(73) Assignee: STAGE ANALYTICS, Suwanee, GA (US)

(21) Appl. No.: 18/669,985

(22) Filed: May 21, 2024

Related U.S. Application Data
(60) Provisional application No. 63/469,114, filed on May 26, 2023.

(51) Int. Cl. G16H 50/80; G06N 3/0442

(52) U.S. Cl. CPC G16H 50/80; G06N 3/0442

(57) ABSTRACT

Many epidemics such as the opioid epidemic, are complex and dynamic, yet relatively little is known regarding its likely future impact and the potential mitigating impact of interventions to address it. The instant systems and methods provide a dynamic decision dynamic open Markov model, configured to provide updated estimates of the future magnitude of an epidemic, and project the potential association of key interventions with mitigation of the epidemic via a redress model and abatement model. This novel approach addresses the deficiencies of conventional approaches to modeling opioid epidemics by granularly analyzing populations via a framework including an open Markov model, redress model, abatement model, and evaluation model, that simulate the impact of interventions on the population, and forecast the remedies and resources required to abate the epidemic.

### CROSS-REFERENCE TO RELATED APPLICATION

This Application claims the benefit of U.S. Provisional Application Ser. No. 63/469,114, filed May 26, 2023, which is incorporated herein by reference in its entirety.

### TECHNICAL FIELD

The present disclosure is generally related to the fields of epidemic modeling, epidemic mitigation strategies, epidemic abatement, and machine learning.

### BACKGROUND

On average, approximately 48,000 individuals in the US died from an opioid overdose per year over the last nine years with available data, and morbidity and mortality from the opioid epidemic continues to accrue. Because of the opioid epidemic's magnitude and scope, it is important to understand as much as possible about how the crisis may evolve, including the interplay of factors accounting for injuries and deaths. It is also important to understand the potential impact of measures designed to avert future harms. While no conventional epidemiologic model can capture every aspect of the epidemic, formal models allow for systematic analysis of previous research and the explicit and quantitative estimation of the impact of different interventions. Models also can assist in comparing short-term outcomes with long-term outcomes, examining the impact of interventions in subpopulations of interest, and quantifying the economic, as well as public health, costs, and benefits of different approaches to abate opioid-related harms.

To fully estimate the epidemic's scope and the impact of interventions to address it, it is essential to consider differences in individuals using prescription opioids vs heroin or illicit fentanyl, the increased risk of second overdose in people who have experienced an initial overdose, and the evolving time-dependent nature of the epidemic. Despite the contributions of prior models of the epidemic, most have not incorporated these elements, nor have they accounted for the more than 2.5 million individuals in the US who report lifetime—but not past year—opioid use disorder (OUD) In addition, earlier models have tended to regard treatment of OUD as a single entity rather than differentiating among different phases of treatment or recovery to allow for flexible modeling of different subpopulations, such as those receiving more or less intensive care.

Conventional approaches to epidemic modeling have been limited by their inability to process and analyze the vast amounts of data points, each potentially encompassing thousands of individual records, that are indicative of the complex and dynamic nature of epidemics such as the opioid crisis. These traditional models often lack the computational framework to handle the granularity of data that is now available, which includes a multitude of variables and parameters that can influence the trajectory of an epidemic. This limitation hinders the ability to make accurate, real-time predictions and to evaluate the potential impact of various intervention strategies across different population segments and geographic regions.

Furthermore, existing models have struggled to provide real-time determinations due to the computational intensity and complexity of processing large-scale data sets. The ability to make timely decisions is paramount in responding to an epidemic, where delays can result in increased morbidity and mortality. The lack of real-time processing capabilities in conventional models has therefore been a substantial barrier to implementing timely and effective intervention strategies. This has underscored the urgent need for a more robust modeling framework that can leverage advanced computational techniques, including artificial intelligence and machine learning, to assimilate and analyze data at the scale and speed that modern epidemics demand. Given these challenges, there is a need for systems and methods that address the deficiencies of the aforementioned conventional approaches.

### BRIEF DESCRIPTION OF THE FIGURES

FIG. 1 illustrates a computing environment, according to various embodiments of the present disclosure.

FIG. 2 illustrates an open Markov model method for projecting future states of populations, according to various embodiments of the present disclosure.

FIG. 3 illustrates a redress model method for simulating the impact of remedies in abating an epidemic, according to various embodiments of the present disclosure.

FIG. 4 illustrates an abatement model method, according to various embodiments of the present disclosure.

FIG. 5 illustrates a system flow diagram, according to various embodiments of the present disclosure.

FIG. 6 illustrates a machine learning architecture, according to various embodiments of the present disclosure.

FIG. 7 illustrates an interface, according to various embodiments of the present disclosure.

FIG. 8 illustrates a block diagram for a computing device, according to various embodiments of the present disclosure.

### SUMMARY

The present disclosure introduces a transformative system and method for dynamic epidemic modeling and abatement, particularly addressing the opioid crisis. This innovation represents a substantial improvement over conventional approaches by overcoming their deficiencies and providing a technical advancement in the field of epidemic management and abatement. Conventional models have been limited in their ability to adapt to the evolving nature of epidemics and often fail to account for the complex interplay of factors influencing their progression. The disclosed systems and methods solve these issues by employing a cross-communicative multi-model approach that enables real-time complex computations and integrates machine learning techniques.

The disclosed systems and methods provide a dynamic open Markov model configured to provide updated estimates of the future magnitude of an opioid epidemic, and project the potential association of key interventions with mitigation of the epidemic via a redress model and an abatement model. This novel approach addresses the deficiencies of conventional approaches to modeling opioid epidemics by granularly analyzing populations via a framework including an open Markov model, redress model, and abatement model, that simulate the impact of interventions on the population, and forecast the remedies and resources required to abate the epidemic. In some embodiments, the framework may include various techniques for predicting and forecasting through artificial intelligence and machine learning. Notably, although this disclosure discusses examples related to opioid modeling and abatement, one having skill in the art will appreciate that the disclosed techniques may be applied to other relapse-related diseases, large-scale public health issues (e.g., tobacco and vaping epidemic), and disorder-related epidemics.

In one embodiment, the implementation of these novel systems and computer-implemented methods may involve one or more processors and a non-transitory computer-readable medium storing instructions, wherein the one or more processors are configured to implement instructions for: aggregating population data and epidemic data from one or more external databases. The implementation may further include inputting the one or more populations as input for an open Markov model and identifying a set of predetermined time-dependent states, wherein a set number of the predetermined time-dependent state are associated with epidemic interventions. This approach may additionally include identifying an initial state from the set of predetermined time-dependent states for each of the one or more populations and assigning, for each state of the set of predetermined time-dependent states, a transition probability to the one or more populations. The one or more processors may then project, by the open Markov model for a specific future point in time, a projected state of the number of time-dependent states, for each of the one or more populations and generate an intervention trend ratio by comparing the one or more populations at their respective initial state to the one or more populations at their projected state. Notably, the open Markov model works in conjunction with one or more additional models.

As such, the one or more process may receive the projected state for each of the one or more populations as input for a redress model. The implementation may further include identifying remedies for abating an epidemic in a specific local geographic region, wherein the remedies include one or more resources and respective resource values associated with the specific local geographic region and simulating, via the redress model, an impact of the resources in abating the epidemic in the specific local geographic region. This approach may further require determining a final intervention trend ratio. The outcome of the redress model may be used by certain downstream processes, such as an abatement model.

Here, the one or more processors may receive the one or more resources and respective resource values from the redress model as input for an abatement model. The abatement model may determine an aggregate value associated with implementing each of the one or more resources in abating the epidemic in the specific local geographic region, and further determine a yearly value by applying an inflation value to the aggregate value. The abatement model may be configured to leverage the aforementioned determinations to project a yearly value for each year within a predetermined number of future years.

Notably, this embodiment may leverage one or more systems, computer-implemented methods, and/or non-transitory computer-readable mediums.

### DETAILED DESCRIPTION

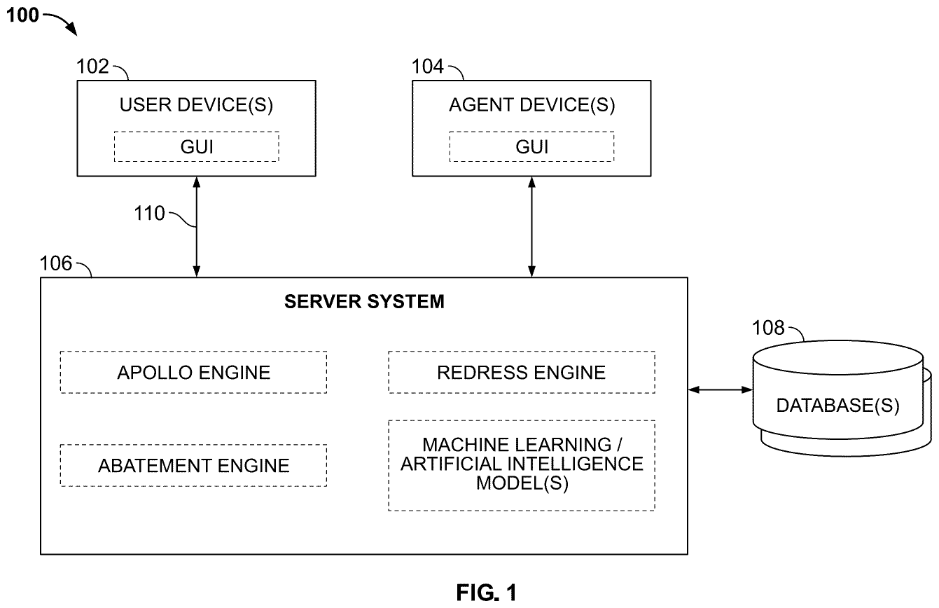

Referring to FIG. 1, according to embodiments of the present disclosure, computing environment 100 may implement a framework for the real-time systemic analysis of population and epidemic data, comparing short-term outcomes with long-term outcomes, examining the impact of interventions in subpopulations of interest, and quantifying the economic, as well as public health, costs, and benefits of different approaches to abate opioid-related harms. Computing environment 100 may include one or more user device(s) 102, agent device(s) 104, and one or more database(s) 108, communicatively coupled to server system 106, and further configured to communicate through network 110. Notably, FIG. 1, while depicted in the context of abating opioid-related harms, serves as an illustrative example and is not restrictive. For example, computing environment 100 may be used in relation to abating tobacco and vaping related harms.

In one or more embodiments, user device(s) 102 and agent device(s) 104 are operated by a user. Users may include, but are not limited to, individuals such as, for example, software engineers, subscribers, clients, prospective clients, government entities, think tanks, municipalities, judicial organizations, or customers of an entity associated with server system 106, such as individuals who have obtained, will obtain, or may obtain a product, service, provided by an entity associated with server system 106.

User device(s) 102 and agent device(s) 104 according to the present disclosure may include, without limitation, any combination of mobile phones, smart phones, tablet computers, laptop computers, desktop computers, server computers or any other computing device configured to capture, receive, store and/or disseminate any suitable data. In one embodiment, a user device(s) 102 includes a non-transitory computer readable medium (e.g., non-transitory memory) including machine readable instructions, one or more processors for executing the instructions, a communications interface that may be used to communicate with server system 106, (and, in some examples, with the database(s) 108), a user input interface for inputting data and/or information to the user device and/or a user display interface for presenting data and/or information on the user device. In some embodiments, the user input interface and the user display interface are configured as an interactive graphical user interface (GUI). These devices are also configured to provide server system 106, via the interactive GUI, with input information for further processing. In some examples, the user input interface and the user display interface are configured as an interactive GUI. These devices are also configured to provide server system 106, via the interactive GUI, input information (e.g., queries, questions, prompts, commands, and code) for further processing. In some embodiments, the interactive GUI is hosted by server system 106 or it can be provided via a client application operating on user device(s) 102 and/or agent device(s) 104.

The server system 106 includes one or more processors, servers, databases, communication/traffic routers, non-transitory memory, modules, and interface components. In one or more embodiments, server system 106 hosts, stores, and operates a one or more engines/models, including but not limited to an apollo engine, redress engine, abatement engine, and machine learning/artificial intelligence model(s), to analyze populations affected by an epidemic, implement one or more models configured to simulate, forecast and predict remedies and resources required to abate an epidemic. Server system 106 may receive/implement one or more models of modeling an epidemic in response to user input data via an interface, in response to an API call, or an automatic predetermined interface workflow, a user input query and/or in response to a series of prompts pushed to various computing devices in computing environment 100.

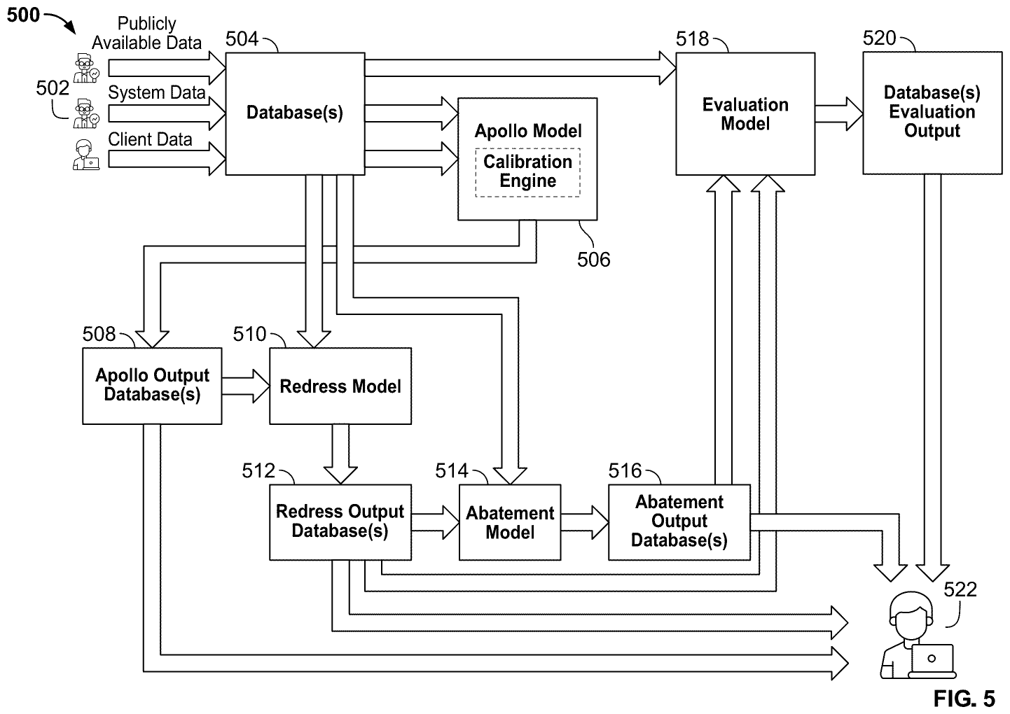

Database(s) 108 may be a cloud-based collection of organized data stored across one or more storage devices. Database(s) 108 may be complex and developed using one or more design schema and modeling techniques. Database(s) 108 may be hosted at one or more data centers operated by a cloud computing service provider. The database(s) 108 and may be geographically proximal to or remote from the server system 106 and configured for maintaining/storing: training data, population data, geographic data, epidemic data, and the like. The database(s) 108 and may be in communication with server system 106, end user device(s) 102, agent device(s) 104 via network 110. Database(s) 108 stores various (encrypted) data, including user activity data, user preferences data, litigation related data, redress data, personnel data, and artificial intelligence/machine learning training data that can be modified and leveraged by server system 106, user device(s) 102, and/or agent device(s) 104. Various data in the database(s) 108 may be refined over time using a machine learning model and/or an artificial intelligence model, for example the machine learning models discussed with respect to FIG. 5. Additionally, database(s) 108 may be deployed and maintained automatically by one or more components shown in FIG. 1.

Network 110 may be of any suitable type, including individual connections via the Internet, cellular or Wi-Fi networks. In some embodiments, network 110 may connect terminals, services, and mobile devices using direct connections, such as radio frequency identification (RFID), near-field communication (NFC), Bluetooth™, low-energy Bluetooth™ (BLE), Wi-Fi™, ambient backscatter communication (ABC) protocols, USB, WAN, LAN, or the Internet. Because the information transmitted may be personal or confidential, security concerns may dictate one or more of these types of connection be encrypted or otherwise secured. In some embodiments, however, the information being transmitted may be less personal, and therefore, the network connections may be selected for convenience over security.

In some embodiments, communication between the elements may be facilitated by one or more application programming interfaces (APIs). APIs of server system 106 may be proprietary and/or may be examples available to those of ordinary skill in the art such as Amazon® Web Services (AWS) APIs or the like.

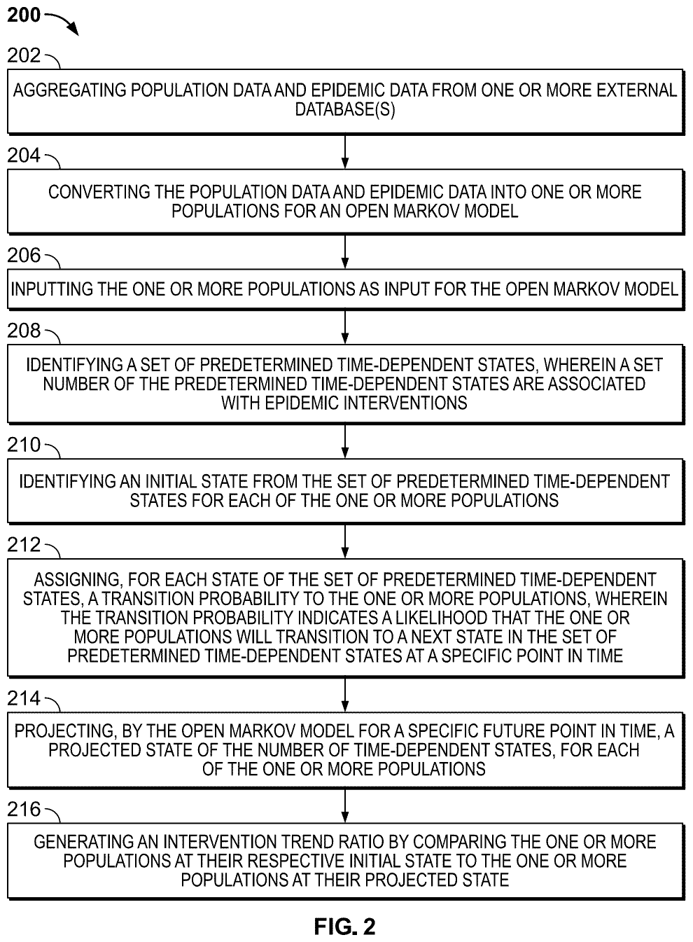

Referring to FIG. 2, an open Markov model method 200 for projecting future states of populations is depicted, according to one or more embodiments of the present disclosure. At 202, server system 106 may aggregate population data and epidemic data from one or more external database(s) 108; in addition, server system 106 may aggregate client data (e.g., population data and epidemic data) from one or more clients operating user device(s) 102 or from database(s) 108 for one or more downstream purposes. In one non-limiting example, server system 106 may implement code configured to crawl or scrape data from one or more databases maintained by third parties (e.g., United States Census Bureau, Centers for Disease Control and Prevention (CDC) Wide-Ranging Online Data for Epidemiologic Research, National Survey on Drug Use and Health (NSDUH), and state and local government entities) and from the National Survey on Drug Use and Health, National Health and Nutrition Examination Survey, and National Epidemiologic Survey on Alcohol and Related Conditions-III. Here, the aggregated population data and epidemic data may be received and/or scraped in response to a user query (e.g., from the user device(s) 102 or agent device(s)) or pulled according to a predetermined schedule. The population data and epidemic data may include, but is not limited to, national general population data, such as the total population in a specific geographic region (e.g., the United States) on a year-by-year basis and corresponding demographic data (e.g., age, ethnicity, occupation, birth, death, opioid user per capita, treatment data, prescription data, hospital admission data, and the like). National data related to opioid overdose deaths, non-fatal opioid overdoses, opioid prescription, and opioid prescription trend data. In another embodiment, related to abating tobacco and vaping harms, data may be aggregated from relevant sources storing data associated with tobacco and vaping usage.

A specific local geographic region may also be selected, wherein local population data and epidemic data may be aggregated regarding that specific local region (e.g., a city). For example, server system 106 may receive data for the city of Washington, D.C. or for the mid-Atlantic region. Here, server system 106 may implement code configured to crawl or scrape data from one or more databases maintained by local third parties, such city/state/region municipalities, governmental agencies, or third parties maintaining population and epidemic related data, associated with providing resources to, or monitoring the societal needs of, Washington, D.C. or for the mid-Atlantic region.

However, in some instances data may not be available for a specific local geographic region. In this instance, data can be extrapolated from national-level data to estimate the necessary data for a specific local geographic region level data. For example, in an instance where data for a specific local geographic region is unavailable as it relates to opioid overdose deaths, server system 106 may determine the number of people residing in particular country on a national level (e.g., population of the United States) and divide it by the number overdoses at the national level. Here, server system 106 may be configured to leverage the known population for a specific local geographic region (e.g., the population of Washington, D.C.) and thereby calculate the cross product of the unknown overdose deaths in the specific geographic region. In this instance, since a specific local geographic region accounts for a certain percentage of a national population, the server system 106 is estimating that the specific local geographic region likely also accounts for a proportional amount of overdose deaths.

At 204, server system 106 may convert the population data and epidemic data into one or more populations for an open Markov model (i.e., an Apollo model). For example, server system 106 may parse the population data for relevant information, such as the number of people in a given country or region and the number of people affected or using opioids (and their corresponding demographic information). The population data and epidemic data may be converted from a first format to a second format unique to computing environment 100 and server system 106. Further, the data may be converted (e.g., into token and vector format) and manipulated, such that it can be used as input for one or more models (e.g., an open Markov model implemented by the Apollo engine) as one or more populations. In some instances, converting the data from a first format to a second format may require various encryption and decryption techniques.

At 206, the one or more populations may be inputted in the open Markov model. Here, the open Markov model may be implemented by the Apollo engine hosted by server system 106. The open Markov model may be designed and configured to capture associations from the one or more populations underpinning the epidemiologic nature of the opioid epidemic. The open Markov model may consist of 32compartments distinguishing 7 major populations: (1) no opioid use (general population in FIG. 1); (2) prescription opioid medical use; (3) prescription opioid nonmedical use; (4) use of heroin, illicit fentanyl, or other illicit opioids; (5) opioid use disorder (OUD) from prescription opioids; (6) heroin use disorder (HUD) with prior prescription opioid use (HUD-Rx); and (7) heroin use disorder without prior prescription opioid use (HUD-NonRx). The model also provides for seven (7) subpopulations with prescription OUD, HUD-Rx, and HUD-NonRx based on degrees of clinical stability and treatment engagement. In a non-limiting capacity, the open Markov model may further consist of 109 periodic (e.g., monthly) transitions between the 32 compartments.

At 208, server system 106 may identify a set of predetermined time-dependent states, wherein a set number of the predetermined time-dependent states are associated with epidemic interventions. In addition, the predetermined time-dependent states may further include states indicative of increased or ongoing use of opioids. These further include 25 time-dependent transitions reflecting changes in the epidemic, such as increased use of fentanyl and increasing lethality of opioids, decreased prescribing of opioid medications, increased access to medications for addiction treatment (MAT), and population growth including but not limited to in the United States. The open Markov model interventions may include but are not limited to six (6) categories, such as reducing prescription opioid oversupply, increasing MAT uptake, increasing detox success, reducing MAT discontinuation rate, increasing naloxone distribution and user for prescription opioids only, and increasing naloxone distribution and user for illicit opioids. Indeed, the interventions may vary depending on the epidemic that is being analyzed.

At 210, server system 106 may identify an initial state from the set of predetermined time-dependent states for each of the one or more populations. For example, server system 106 may apportion the one or more populations amongst the predetermined time-dependent states based on the data gleaned from the at 202-204.

At 212, server system 106 may assign, via the open Markov model, for each state of the set of predetermined time-dependent states, a transition probability to the one or more populations, wherein the transition probability indicates a likelihood that the one or more populations will transition to a next state in the set of predetermined time-dependent states at a specific point in time. Here, the probabilities may be calculated based on historical data gleaned from reported data gleaned from external data sources, such as the CDC and NSDUH. For example, server system 106 may calculate a transition probability by referencing a particular individual's (or an entire sub-population's) reported state for a specific previous year, in comparison to that individual's reported state for the immediate following year. Server system 106 may perform a similar calculation for each predetermined time-dependent state, such that in aggregate, server system 106 can determine an individual's, a sub-population, or an entire population's propensity to transition to another predetermined time-dependent state. One having skill in the art can understand that other transitions and their corresponding probabilities may vary depending on the specific population being assessed and the epidemic being analyzed.

At 214, server system 106 may project, by the open Markov model, for a specific future point in time, a projected state of the number of time-dependent states, for each of the one or more populations. Here, given the transition probabilities determined at 212, the open Markov model may be configured to run a simulation such that various transitions can determine for the one or more populations for a particular date in the future (e.g., days, months, and/or years in the future). The particular date in the future may be a predetermined date, a date determined to be relevant by server system 106 based on calculations by the open Markov model, and/or date set by a user operating user device(s) 102 or agent device(s) 104. Notably, the open Markov model, may be configured to project multiple different outcomes. For example, the open Markov model may be configured to simulate the impact of not implementing any interventions, in addition to implementing some of the interventions, or all of the interventions. Such projections can be used to explicitly convey the impact of pursuing one opioid abatement strategy over another. To enhance the accuracy of these projections, the open Markov model may be calibrated by comparing predicted values, such as a predicted state of a population for a given time period, with known values, such as the actual observed state of the population for the same period. This calibration process adjusts the model's parameters to enhance performance accuracy. By quantifying the difference between predicted and known values, the open Markov model refines its algorithms to improve the precision of future projections, ensuring accurate and reliable estimations of epidemic trajectories and the effectiveness of intervention strategies.

At 216, server system 106 may generate an intervention trend ratio by comparing the one or more populations at their respective initial state to the one or more populations at their projected state. One key metric, here, is the percentage of the one or more populations in active OUD, as the goal for most governments, governmental agencies, and municipalities, is to transition populations into survivorship status via treatment or prevent populations from entering into OUD through various preventative measures. As such, active OUD is tracked to show the progress of populations transitioning into preferred states (i.e., survivorship and not entering into OUD). As such, server system 106 may generate an intervention trend ratio that is indicative of the percentage of population (or how many individuals) that are in active OUD at a present or future date in comparison to past date. Here, server system 106 may compare the initial state determined at 210 to the projected sate at 214 to generate the intervention trend ratio. In another embodiment, a similar process may be implemented to generate an intervention trend ratio associated with tobacco and vaping epidemics.

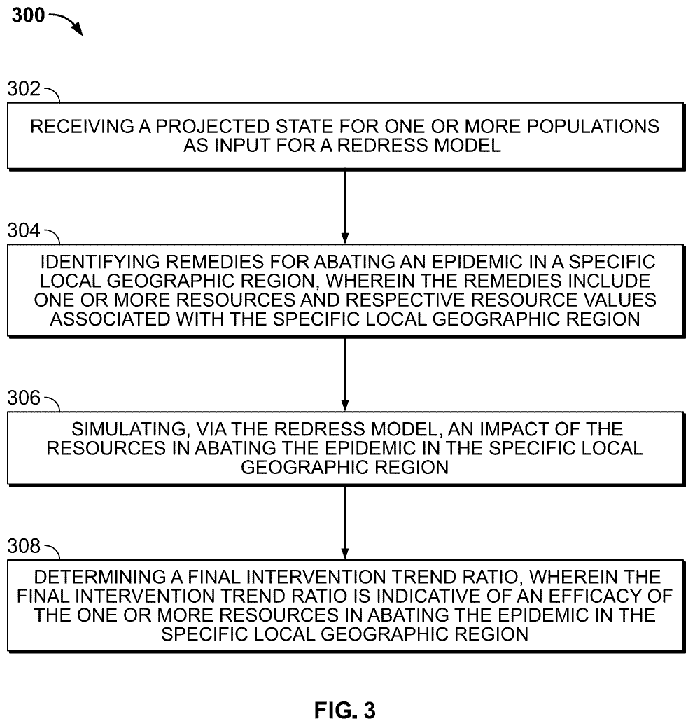

Referring to FIG. 3, a redress method 300 for simulating the impact of remedies in abating an epidemic is depicted, according to one or more embodiments of the present disclosure. At 302, server system 106 may receive projected state for each of the one or more populations as input for a redress model. For example, the open Markov model described in relation to FIG. 2 may pass the projected state of the number of time-dependent states for each of the one or more populations as input for a redress model method.

At 304, server system 106 may identify, by a redress model, remedies for abating an epidemic in the geographic region, wherein the remedies include one or more resources and respective resource values associated with the specific local geographic region. For example, the redress model may determine the remedies available in the specific local geographic region that are available to equitably address the needs of the population affected by the epidemic in the specific local geographic region. Remedies may include but are not limited to prevention, intervention, recovery, and addressing location specific needs related approaches; for example, specific remedies may health professional education, patient and public education, personnel, safe storage and drug disposal, harm reduction, leadership surveillance and implementation, connecting individuals to care, treatment for opioid user disorder, managing complications attributable to the epidemic, workforce expansion and resiliency, distributing naloxone and providing training, public safety, criminal justice, vocational training, education, and job placement, mental health, and the like. Remedies may be selected based on input from a user operating user device(s) 102 and agent device(s) 104, automatically selected by server system 106, and/or based on previously simulated models of similarly situated specific local geographic regions. While the aforementioned remedies may, in some respects, be unique to opioid-related harms, in another embodiment, server system 106 can be configured to identify remedies related tobacco and vaping.

At 306, server system 106 may simulate, via the redress model, an impact of the resources in abating the epidemic in the specific local geographic region. For example, in response to receiving the input at 302 and identifying remedies for abating the epidemic occurring in the specific local geographic region at 304, the redress model may simulate the one or more outcomes of the identified remedies being implemented in the specific local geographic region. As discussed in relation to 214, a projected state of one or more populations may be simulated and determined be the open Markov model. Notably, the simulated implementation of the remedies on the one or more populations may impact the projected state of the one or more populations, such that the efficacy of the remedies may be quantitatively apparent. For example, the redress model is configured to simulate the percentage of the one or more populations that are transitioned to ideal states, such as into survivorship and/or prevented from entering OUD. Further, the redress model is configured to quantitatively determine how many resources, such as doctors, clinics, doses of pharmaceuticals, treatment programs (and the like), that may be required to transition the one or more populations to the aforementioned ideal states.

At 308, server system 106 may determine a final intervention trend ratio, wherein the final intervention trend ratio is indicative of the efficacy of the resources in abating the epidemic in the specific local geographic region. In some embodiments, the one or more populations cannot be benchmarked based on the open Markov model. As such, here, the server system 106, via the redress model, is configured to evaluate the quantitative impact of the remedies on the one or more populations at a particular point in time (e.g., 1-15 years in the future) by determining a change in the states (e.g., the percentage of a population that has transitioned) of the one or more populations at the particular point in time in comparison to an earlier date (e.g., the initial state discussed at 210). Notably, the final intervention trend ratio reflects both the impact of the interventions discussed in relation to the open Markov model in the description of FIG. 2 and the remedies discussed above in relation to FIG. 3.

Given, that the remedies and resources leveraged in the redress model are, in some instances, programs, personnel, clinics and the like, the redress model is configured to quantitatively depict the number of said remedies and resources necessary to bring about the change indicated by the redress model. Further, in some instances, it may be determined that a large percentage of the total resources may be required in the early years (e.g., years 1-5 out of a 15-year epidemic abatement plan). As such, the redress model may apply a logarithmic algorithm to the implementation of the resources, such that more resources are allocated in the earlier years of an epidemic abatement pan in order to have a greater impact sooner in the specific local geographic region. As previously discussed, server system 106 may be configured address other relapse, disease, and epidemic related issues. As such, in another embodiment, server system 106 may be configured to implement similar steps as those discussed in relation to 302-308 above to address issues related to tobacco and vaping, for example.

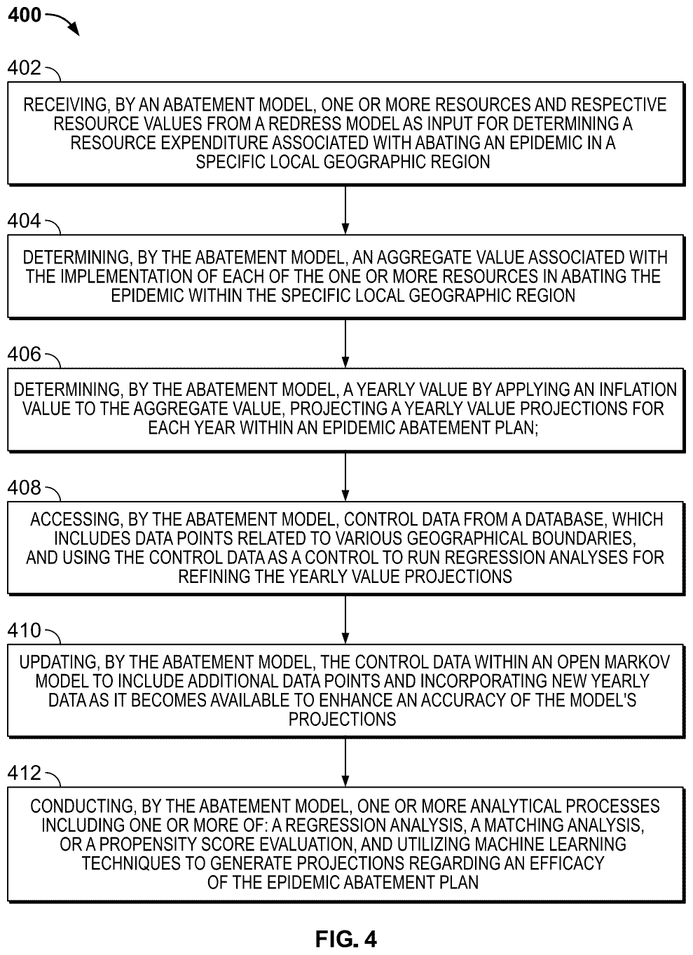

Referring to FIG. 4, an abatement model method 400 for projecting a yearly value associated with abating an epidemic is depicted, according to various embodiments of the present disclosure. Step 402, may involve receiving, by the abatement model, one or more resources and respective resource values from the redress model as input for determining a resource expenditure associated with abating an epidemic in a specific local geographic region. Here, the abatement model may receive an estimated or exact number of resources determined to be required by the redress model (as discussed in relation to FIG. 3) to effectively abate the epidemic in the specific local geographic region. The abatement model will also receive the costs (i.e., the value of money required to produce a remedy) associated with each respective remedy (e.g., each vehicle, clinic, personnel, etc.).

Step 404, may involve determining, by the abatement model, an aggregate value associated with the implementation of each of the one or more resources in abating the epidemic within the specific local geographic region. For example, the abatement model is configured to multiply the quantity of each resource by the resource value (i.e., the cost), and then add the cost of each resource to determine a total aggregate value of all the resources.

Step 406, may involve determining, by the abatement model, a yearly value by applying an inflation value to the aggregate value, projecting yearly value projections for each year within an epidemic abatement plan. This may involve applying an inflation value to an aggregate value enables the abatement model to account for the potential increase in the cost of living/acquiring goods/transacting business year-over-year during the implementation of an epidemic abatement model in a specific local geographic region. For example, for each year in the epidemic abatement plan for a specific local geographic region, the abatement model may determine the total cost in implementing the epidemic abatement plan as discussed above at 404, and further apply an inflation value (e.g., consumer price index (CPI)) to the aggregate value of all resources required. The yearly value will reflect the cost of implementing the remedies in a specific year and further accounts for the rising cost of inflation during that specific year.

The abatement model is configured to determine a yearly value (i.e., inclusive of cost of each resource while accounting for inflation) of implementing an epidemic plan with the remedies and resources discussed in the redress model, for every year in an epidemic abatement plan. As such, the abatement model may be further configured to automatically project the yearly value for implementing an epidemic abatement plan or project the yearly value in response to an input/request from a user. Although 402-408 are discussed in relation to an opioid epidemic, in another embodiment, the abatement model may be configured to project the yearly value for implementing a tobacco/vaping epidemic abatement plan. Although this example is discussed in relation to yearly value projections, one having skill the art will appreciate that projections can be according to any time, that is, on a daily, monthly, quarterly, or yearly basis.

Step 408, may involve accessing, by the abatement model, control data from a database, which includes data points related to various geographical boundaries, and using the control data as a control to run regression analyses for refining the yearly value projections. Here, the abatement model is configured to provide accurate resource forecasts for epidemic abatement efforts. By incorporating control data that reflects the economic conditions and resource distribution across different geographical areas, the abatement model may adjust its projections to account for regional variations in costs and resource availability. The regression analyses may utilize this control data to identify trends and correlations that can refine the abatement model's predictions, ensuring that the yearly value projections are not just based on historical data but are also sensitive to the geographical nuances that can affect the cost and effectiveness of the interventions. This level of detail is particularly valuable for policymakers and public health officials who require precise budgetary estimates to plan and allocate resources effectively. The control data may include, but is not limited to, regional cost of living indices, healthcare resource distribution metrics, local economic growth rates, and demographic shifts that can impact the demand for and cost of epidemic abatement resources. By continuously updating the control data and incorporating the latest information into the regression analyses, the abatement model remains dynamic and responsive to changing conditions, thereby enhancing the reliability of its financial projections for epidemic management.

Step 410 may involve updating, by the abatement model, the control data within the open Markov model to include additional data points and incorporating new yearly data as it becomes available to enhance the accuracy of the model's projections. This step may ensure that the abatement model's projections are as accurate and current as possible. The abatement model may be configured to incorporate additional data points into the control data, which may include new epidemiological findings, updated intervention efficacy rates, or changes in population demographics. By integrating these additional data points, the model can adjust its projections to reflect the latest understanding of the epidemic's dynamics.

Moreover, the abatement model may be tasked with incorporating new yearly data as it becomes available. This includes data may include the actual costs incurred, the outcomes of interventions implemented, and any shifts in the epidemic's trajectory. The inclusion of this new yearly data may allow the abatement model to calibrate its projections, ensuring that they align with real-world observations and outcomes.

The continuous updating of control data may be a dynamic process that allows the open Markov model to evolve with the epidemic. It enables the model to learn from the past and adjust its forecasts for the future, thereby providing decision-makers with projections that are both informed by historical trends and adaptable to new developments.

Step 412 may involve conducting, by the abatement model, one or more analytical processes including one or more of: a regression analysis, a matching analysis, or a propensity score evaluation, and utilizing machine learning techniques to generate projections regarding the efficacy of the epidemic abatement plan. These analytical processes may enable the abatement model to provide a nuanced evaluation of the potential impact of the epidemic abatement plan. By utilizing machine learning techniques, the abatement model can analyze complex datasets to identify patterns, trends, and relationships that may not be immediately apparent through traditional analysis methods.

Regression analysis may be employed to understand the relationship between the various interventions and their impact on the epidemic's progression. This statistical method allows the abatement model to estimate the effect size of each intervention, providing a quantitative measure of their effectiveness. Matching analysis may be used to compare similar groups within the population data, ensuring that the impact of interventions is assessed within comparable cohorts, thereby reducing bias in the evaluation. Propensity score evaluation is a technique that may be used assess the probability of intervention assignment based on observed characteristics, allowing for a more accurate estimation of the intervention effects by accounting for confounding variables.

The integration of these analytical processes within the abatement model allows for a comprehensive evaluation of the epidemic abatement plan's potential impact. The insights gained from these analyses are invaluable for decision-making, as they provide a data-driven basis for assessing the effectiveness of interventions and for planning future strategies.

Referring to FIG. 5, a system flow diagram 500 is depicted according to various embodiments of the present disclosure. The system flow diagram 500 illustrates the integration of various components and data flow within the epidemic management system. The system flow diagram 500 begins with system input data 502, which represents the initial data inputs that may include but is not limited to, past, present, and predicted, population data, epidemic data, and other relevant information. This data may be sourced from external databases such as public health records, surveys, and other data repositories, from clients (e.g., governments, municipalities, cities, states, counties, and the like), and from preexisting system data, and fed into database(s) 504, which may include one or more separate databases to store each data type from each source separately.

The Apollo model 506 may be configured to process input data 502 received from database(s) 504. It utilizes an open Markov model framework to simulate the progression of an epidemic, dynamically representing various states within a population and the transitions between these states over time. The model receives population data and epidemic data from the database(s) 504, including demographic information, infection rates, recovery statistics, and intervention details, which are used to populate the model's state space and parameterize the transitions between states.

Upon processing the input data, the Apollo model 506 may be configured to identify and assign transition probabilities to each state within the set of predetermined time-dependent states. These probabilities are indicative of the likelihood of the population transitioning from one state to another, as informed by the associated interventions and historical data. The model may be configured to project the future state of the population at specific points in time, thereby enabling the estimation of the epidemic's trajectory under various intervention scenarios.

The output generated by the Apollo model 506, which may include the projected states of the population and the intervention trend ratios, may be stored in the Apollo output database(s) 508. This output may be used for subsequent modeling steps, such as the redress model 510 and the abatement model 514, which may be configured to further refine the analysis of interventions and their impact on abating the epidemic. The Apollo model 506 may be configured for providing updated estimates of the future magnitude of an epidemic and in projecting the potential association of interventions with its mitigation.

Additionally, the Apollo model 506 may be configured to calibrate its predictive capabilities by comparing predicted values, such as a predicted state of a population for a given time period, with known values, such as the actual observed state of the population for the same period. This calibration process may be configured to adjust the model's parameters to enhance performance accuracy. By quantifying the difference between predicted and known values, the Apollo model 506 may be configured to refine its algorithms to improve the precision of future projections, ensuring accurate and reliable estimations of epidemic trajectories and the effectiveness of intervention strategies.

The redress model 510 may receive the Apollo model output (e.g., projected state of the populations from the Apollo output database(s) 508) and simulate the impact of various remedies and resources on abating the epidemic within a specific local geographic region. as discussed in relation to the description of FIG. 3. The outcomes of the redress model 510 may be stored in the redress output database(s) 512.

The abatement model 514 utilizes the data from the redress output database(s) 512 to calculate the costs associated with implementing the remedies over time. The abatement model 514 takes into account factors such as resource values and inflation to project a yearly value for each year within a predetermined number of future years, as discussed in relation to the description of FIG. 4. The results may be stored in the abatement output database 516.

The evaluation model 518, as component of the system flow diagram 500, is designed to assess the effectiveness and efficiency of epidemic management strategies by processing large-scale data inputs, which may range from thousands to millions of records. The evaluation model 518 is equipped to manage outputs from the Apollo model 506, redress model 510, and abatement model 514, in addition to directly receiving voluminous input from clients on the execution of their epidemic abatement plans. The evaluation model 518 is capable of performing real-time analysis, generating a progress metric that measures the degree to which client implementations align with the recommended epidemic abatement strategies, a recommendation metric that measures the degree to which specific resources need to be adjusted to align with epidemic abatement strategy goals and objectives. This real-time functionality enables the evaluation model 518 to provide immediate feedback on the efficacy of interventions, considering both direct and indirect effects on the populations in question. The generated metrics, including the progress and recommendation metrics, are stored in the evaluation outcomes database(s) 520, which acts as a dynamic repository for continuously updated data, thereby supporting the ongoing optimization of epidemic management strategies. This real-time assessment capability of the evaluation model 518 is a technical feature that supports decision-making, resource distribution, and the system's ability to respond promptly to changes in epidemic conditions. The user device 522 may be operated by a user that operates/owns system flow diagram 500 or a client thereof, and may include a graphical user interface through which users can interact with the system, input data, select models, and view the results of the simulations. The user device 522 may enable users to effectively manage and respond to the epidemic.

The system flow diagram 500 exemplifies the complex interplay between various models and databases, highlighting the system's capability to process and analyze large volumes of data to inform decision-making in epidemic management.

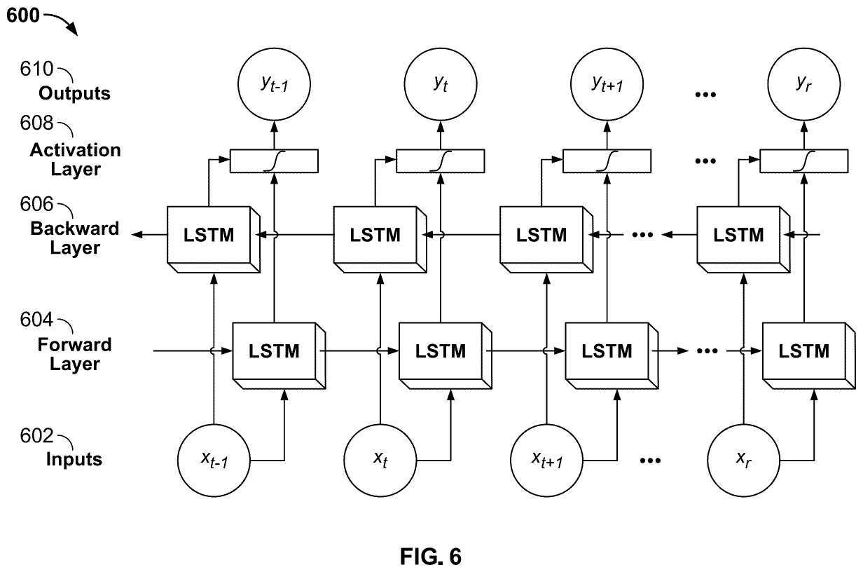

17. Referring to FIG. 6, the machine learning architecture incorporates a bi-directional Long Short Term Memory (Bi-LSTM) network 600, which is a sophisticated form of recurrent neural network (RNN) designed to effectively process sequential data by considering both past and future contexts. The input data sequence 602 may be fed into the network and simultaneously processed by two distinct LSTM layers: the forward LSTM layer 604 and the backward LSTM layer 606.

In machine learning, time series machine learning techniques attempt to forecast a target value based solely on a known history of target values. Similar time series forecasting challenges are present in generating epidemic model projections. In the realm of artificial intelligence/machine learning long short term memory networks are particularly well-suited for forecasting and making predictions based on time series related data. As such, in a non-limiting capacity, the artificial intelligence/machine learning architecture depicted in FIG. 4 may be a recurrent neural network (RNN), specifically a bi-directional long short term memory (LSTM) network. In this instance, the Bi-LSTM is configured to learn all the previous relevant knowledge seen by the network while simultaneously forgetting irrelevant data, especially as it relates to time-series forecasting data. Here, the Bi-LSTM architecture is configured to enable the neural network to store and analyze data both backwards and forwards, such that two hidden layers of opposite directions connect to the same output.

In preparation for input into the Bi-LSTM model, data conversion may be a multi-step process that involves several stages of transformation and normalization. Initially, for example, raw population data and epidemic data are aggregated from various external databases, which may include demographic information, infection rates, recovery statistics, and intervention details. This data is often heterogeneous in format and scale, necessitating a series of preprocessing steps to ensure compatibility with the Bi-LSTM network.

The first step in data conversion may be data cleaning, which involves removing or correcting inaccuracies, handling missing values, and resolving inconsistencies. This step ensures that the data fed into the model is accurate and reliable. Following data cleaning, categorical variables are encoded into numerical format through techniques such as one-hot encoding or label encoding, as neural networks require numerical input (e.g., tokens, vectors, and embeddings).

The next step may involve data normalization or standardization, where numerical data is scaled to a standard range or distribution. This process may assist in speeding up the learning process and leads to faster convergence during model training. Additionally, normalization techniques such as min-max scaling or z-score standardization may be employed.

Once the data is cleaned, encoded, and normalized, it is then sequenced into time-ordered data points that reflect the temporal nature of the epidemic. As an example, each data point in the sequence may represents a snapshot of the epidemic at a given time, capturing the state of the population and the interventions in place.

The input data sequence 602 is fed into the network and simultaneously processed by two distinct LSTM layers: the forward LSTM layer 604 and the backward LSTM layer 606. The forward LSTM layer 604 processes the input data sequence 602 in a chronological manner, learning from the data as it is presented in the sequence, thereby capturing the temporal dependencies that are based on past events and behaviors. Conversely, the backward LSTM layer 606 processes the same input data sequence 602 in reverse, starting from the end of the sequence and moving backwards, which allows it to learn from future events relative to any point in the sequence. This bidirectional processing enables the network to have a more complete understanding of the sequence data, as it can access information from both before and after any given point in the sequence.

Once the forward LSTM layer 604 and the backward LSTM layer 606 have processed the input data sequence 602, their outputs are combined in an activation layer 608. This layer serves as an integrator that synthesizes the information from both directions, effectively merging the insights gained from the past and future contexts of the data. The activation layer 608 applies a non-linear transformation to the combined data, which helps in capturing complex patterns and relationships within the data that may not be immediately apparent.

The output of the activation layer 608 may then be passed on to produce the output data sequence 610. This output data sequence 610 represents the final result of the Bi-LSTM network's processing, which can include predictions about future states of an epidemic, the likelihood of transitions between different states of a population, or other relevant outcomes based on the learned patterns in the data. As an example, the output of the Bi-LSTM network, after processing the input data sequence 602 through the forward LSTM layer 604 and the backward LSTM layer 606, may be a sophisticated set of predictions that encapsulate the temporal dynamics of the epidemic's progression. The activation layer 608 integrates the information from both LSTM layers and applies a non-linear transformation to produce the output data sequence 610.

In one embodiment, the output data sequence 610 may include a range of predictive insights, such as the probability of a population transitioning from one state to another over time, the expected impact of specific interventions on the epidemic curve, and the potential outcomes of public health policies. As discussed, these predictions are generated for various time points in the future, providing a forward-looking perspective on the epidemic's trajectory.

As another example, the output may predict the likelihood of an increase in infection rates following the relaxation of social distancing measures, or the decrease in new cases as a result of a successful vaccination campaign. It may also forecast the resource requirements for healthcare systems, such as the number of hospital beds or medical personnel that will be needed at different stages of the epidemic.

The output can also include financial projections, estimating the costs associated with implementing various interventions and the economic impact of the epidemic on specific regions. These financial forecasts can be broken down by year, allowing policymakers to plan budgets and allocate funds effectively.

Notably, the Bi-LSTM network's output is not static; as it is continuously updated as new data becomes available in real-time.

The Bi-LSTM network 600 may be trained on a large corpus of epidemic related data. In particular, the training data may be divided into a training data set used for model calibration, and a testing data set used for model verification. The training data may include large sets of epidemic observations from various geographic regions, that are used to train the model to perform a desired action. Using this data, the Bi-LSTM network 600 learns the patterns of association between inputs and outputs, and forms a relationship between the different variables; such as the connections between implementing interventions on a certain population and the likelihood of the population transitioning to another state in response to that intervention. Bi-LSTM network 600 may additionally incorporate a feedback loop allowing it to learn from new data/inputs after being initially trained. The validity of the model is tested on an independent data set not used in model training, referred to as the testing data set. Real-life data may be collected from previously simulated models and from disparate population and epidemic data that is aggregated from third party databases (e.g., as discussed in relation to 202). The Bi-LSTM network 600 ability to learn and improve through refinement is a substantial advancement over conventional systems. It achieves this by iteratively processing the data, learning from the temporal patterns, and adjusting its internal parameters to minimize prediction errors. This iterative learning process is what enables the Bi-LSTM network 600 to form a nuanced understanding of the complex dynamics of epidemics.

Given its ability to iteratively refine itself and improve, one of the primary advantages of the Bi-LSTM network 600 over conventional systems is its capacity to handle the temporal dimension of data. Traditional models may not fully capture the sequential nature of epidemics, where past events can influence future outcomes. The Bi-LSTM network 600, with its dual-directional processing, can learn from both past and future contexts within the data sequence, providing a more comprehensive analysis of the epidemic's progression.

Another advantage is the Bi-LSTM network's 600 ability to continuously refine its predictions as new data becomes available in real-time. This adaptability is a marked improvement over static models, which may not account for new trends or changes in the epidemic landscape. The Bi-LSTM network's 600 feedback loop allows for the integration of new observations, ensuring that the predictions remain relevant and accurate. As such, the calculations involved in training and refining the Bi-LSTM model are numerous and complex, making them impractical, if not outright infeasible, for human computation. The model processes vast amounts of data, performs intricate mathematical operations, and adjusts numerous parameters to optimize its predictions. This level of computational complexity is beyond the scope of human capability, especially considering the speed and efficiency with which the model operates. Moreover, the Bi-LSTM network's 600 ability to process and learn from temporal data sequences requires a level of consistency and precision that is unattainable through manual methods. The Bi-LSTM network's 600 capacity to discern subtle patterns and dependencies within the data is a function of its advanced machine learning algorithms, which are designed to perform tasks that are too intricate and laborious for human analysts.

In one embodiment, the open Markov model may leverage the Bi-LSTM network 600 to enhance its predictive capabilities by incorporating the temporal dynamics inherent in epidemic data. The Bi-LSTM network's 600 dual-directional processing may allow the open Markov model to account for both historical trends and potential future changes in the epidemic's progression, thereby improving the accuracy of its state transition predictions.

As an example, the open Markov model may utilize the output data sequence 610 generated by the Bi-LSTM network 600 as a refined input for simulating the epidemic's future states. This sequence may include probabilities of state transitions that are informed by the temporal patterns learned from the input data sequence 602. By integrating these probabilities, the open Markov model can more accurately project the impact of interventions on the epidemic over time.

The integration of the Bi-LSTM network 600 into the open Markov model involves a feedback loop where the model's projections are continuously updated with new data. As the Bi-LSTM network processes additional data sequences, it refines its predictions, which are then fed back into the open Markov model. This iterative process may ensure that the model remains adaptive and responsive to the latest epidemic trends.

In practice, the open Markov model can use the Bi-LSTM network to simulate various “what-if” scenarios, such as the introduction of new treatments or changes in public health policies. The model can then predict how these scenarios might alter the course of the epidemic, enabling policymakers to evaluate the potential effectiveness of different strategies before implementation.

The redress model may integrate the Bi-LSTM network 600 to simulate the impact of various remedies and resources on abating an epidemic within a specific local geographic region. By utilizing the output data sequence 610 from the Bi-LSTM network, the redress model may incorporate the predictive insights regarding the probability of population transitions and the expected impact of interventions over time. This may allow the redress model to forecast the effectiveness of different remedies in reducing the epidemic's prevalence and severity within the targeted region in real-time.

As an example, the redress model may leverage the temporal predictions provided by the Bi-LSTM network 600 to assess how the introduction of resources such as healthcare facilities, treatment programs, and public health initiatives might influence the epidemic's trajectory. The redress model can estimate the changes in infection rates, recovery rates, and overall public health outcomes as a result of deploying these resources. The integration of the Bi-LSTM network's 600 output enables the redress model to consider both historical data and future trends, ensuring that the simulations are grounded in a comprehensive understanding of the epidemic's dynamics.

The redress model's simulations may be informed by the dual-directional analysis of the Bi-LSTM network, which captures the complex interdependencies between various factors influencing the epidemic. This includes the effects of past interventions, current health policies, and potential future changes in the epidemic's behavior. By incorporating these factors, the redress model may provide a nuanced simulation of the epidemic's response to the proposed remedies, aiding in the strategic planning of resource allocation and intervention deployment.

The redress model's output, which includes the simulated impact of resources and the final intervention trend ratio, may be dynamically updated as the Bi-LSTM network 600 processes new data sequences in real-time. This ensures that the redress model's simulations remain accurate and relevant, reflecting the latest available data and insights into the epidemic's progression.

The abatement model may utilize the Bi-LSTM network 600 to project the resource implications of epidemic interventions over time within a specific local geographic region. By leveraging the output data sequence 610 from the Bi-LSTM network 600, the abatement model may incorporate sophisticated predictions about the temporal dynamics of resource utilization and the associated costs of interventions.

As an example, the abatement model may employ the temporal forecasts provided by the Bi-LSTM network 600 to calculate the yearly financial impact of deploying resources such as medical supplies, personnel, and public health programs. The model can determine the aggregate value of these resources by considering the predicted changes in the epidemic's progression and the timing of intervention implementation.

The integration of the Bi-LSTM network's 600 output allows the abatement model to account for both historical expenditure patterns and anticipated future costs, ensuring that the financial projections are comprehensive and reflective of the evolving nature of the epidemic. This includes the ability to adjust for inflation and other economic factors that may influence the cost of resources over time.

The abatement model's financial projections, which include the determination of a yearly value for implementing the epidemic abatement plan, are dynamically updated as the Bi-LSTM network 600 processes new data sequences in real-time. This ensures that the financial planning remains accurate and can adapt to changes in the epidemic's trajectory or in the cost of resources.

The evaluation model may utilize the Bi-LSTM network 600 to assess the overall effectiveness of the epidemic management strategies implemented. By incorporating the output data sequence 610 from the Bi-LSTM network 600, the evaluation model can analyze the long-term outcomes of interventions and remedies applied within various local geographic regions.

As an example, the evaluation model may leverage the predictive insights provided by the Bi-LSTM network to measure the success of interventions in terms of reducing infection rates, improving recovery rates, and achieving public health goals. The model may examine the predicted versus actual outcomes, using the temporal dynamics captured by the Bi-LSTM network to generate progress and recommendation metrics and to understand the efficacy of the strategies over time.

The integration of the Bi-LSTM network's 600 output may enable the evaluation model to conduct a comprehensive analysis that includes both quantitative and qualitative assessments of the interventions. This analysis may assist in identifying the strengths and weaknesses of the current epidemic management approach, providing a basis for refining and improving future strategies.

The evaluation model's analysis is dynamically updated in real-time as the Bi-LSTM network 600 processes new data sequences, ensuring that the evaluation reflects the latest trends and data in epidemic progression. This dynamic updating may further allow for real-time feedback on the effectiveness of interventions, enabling rapid adjustments to be made as the epidemic evolves.

Although, FIG. 6 is discussed in relation to a Bi-LSTM network 600, one having skill in the art will appreciate that one or more additional machine learning techniques may be utilized. For example, machine learning techniques related to classification, forecasting, clustering, decision trees, and linear models, may be used in furtherance of one of one or more processes discussed in FIGS. 2-4. For example, classification algorithms can be used to categorize regions based on their risk levels, forecasting algorithms can predict the spread of the epidemic over time, clustering can identify patterns within population groups, decision trees can aid in determining the sequence of interventions, and linear models can elucidate the relationships between various epidemiological variables.

The Bi-LSTM network 600 may be in communication with a user interface (e.g., interface 700 infra), which users can seamlessly interact with the machine learning models and manage epidemic-related data. It may enable users to select specific geographic regions for analysis, choose appropriate interventions and treatments, and run simulations to visualize the potential impact of these interventions. The interface also facilitates the exploration and manipulation of data, granting access to various epidemic models, including the open Markov model and the redress model, thus serving as a comprehensive tool for epidemic management.

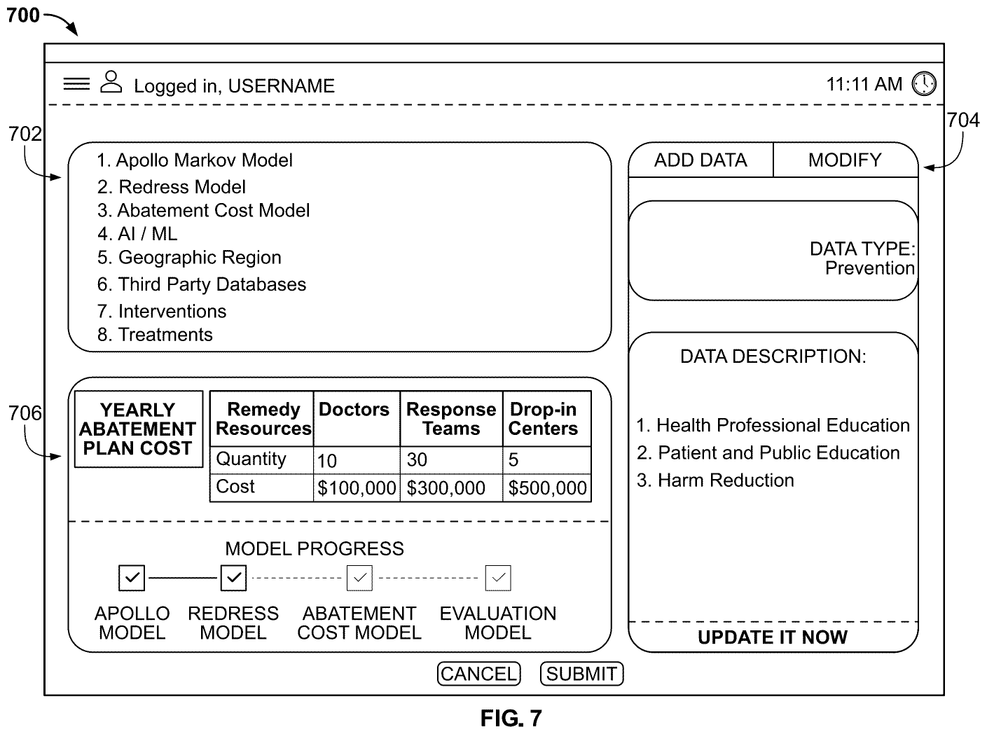

Referring to FIG. 7, an interface 700 is depicted, according to various embodiments of the present disclosure. In some instances, the interface 700 may be an interactive graphical user interface, a stand-alone application, or a sub-feature associated within a software product (e.g., a platform, dashboard and/or website). The interface 700 may be operated by one or more users using user device(s) 102 and/or agent device(s) 104. In some embodiments, interface 700 initiates and plays an integral role for processes simulating the impact of interventions on various populations, and forecasting the remedies and resources required to abate the epidemic impacting those populations, as discussed with respect to FIGS. 2-5. As depicted in FIG. 7, interface 700 includes several dynamic features for implementing one or more epidemic abatement models, manipulating epidemic related data, and running simulations. In the illustrated example, interface 700 includes a user menu region 702, a data manipulation region 704, and dynamic model results region 706.

As depicted in user menu region 702, a series of user options may be populated in response to the type of action being performed by a user. User menu region 702 may additionally be populated with various options based on inputs or changes to data manipulation region 704, and dynamic model results region 706. User menu region 702 may include options for implementing an open Markov model (Apollo), a redress model, an abatement model, artificial intelligence/machine learning (AI/ML) model. User menu region 702 may permit users to select geographic regions, interact with third party databases, manipulate interventions and treatments.

Data manipulation region 704 may enable a user to add/modify data that may be included in a training data set, database, or in a model implemented by server system 106. In particular, data manipulation region 704 may enable a user to customize/calibrate models in order to tailor the models to specific local geographic regions. Notably, the models may be calibrated such that the accuracy of the output of the models fall within a predetermined threshold (e.g., within 5% of third-party published data/observations).

Dynamic model results region 706 may dynamically populate with data based on results from the open Markov model, redress model, and/or cost abatement model. Dynamic model results region 706 may enable real time depiction of transitions of populations between states in the open Markov model. In another instance, the dynamic model results region 706 may depict the yearly value of implementing certain remedies in a specific local geographic region, and the like.

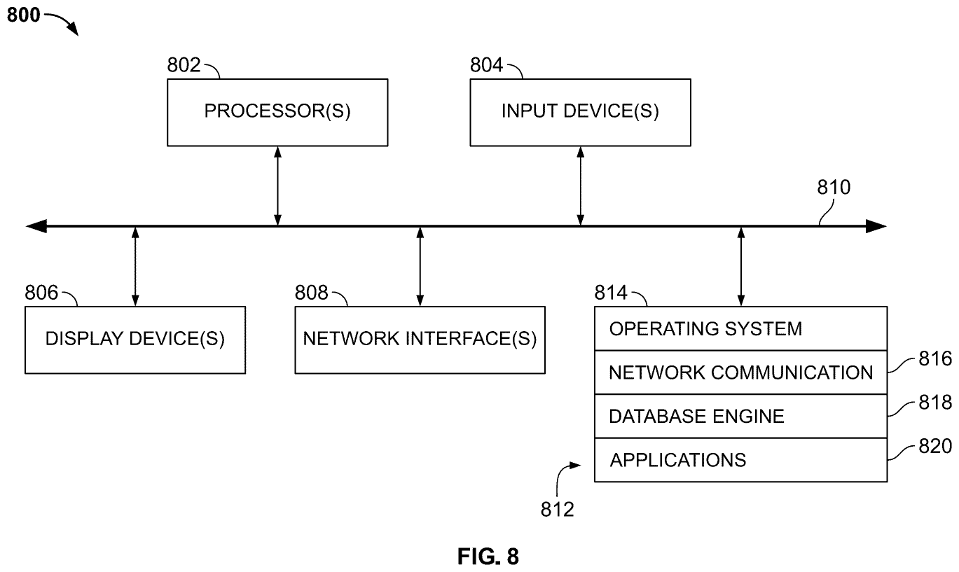

Referring to FIG. 8, a block diagram for a computing device, according to various embodiments of the present disclosure. The computing device 800 may be implemented on any electronic device that runs software applications derived from compiled instructions, including without limitation personal computers, servers, smart phones, media players, electronic tablets, game consoles, email devices, etc. In some implementations, the computing device 800 may include processor(s) 802, (one or more) input device(s) 804, one or more display device(s) 806, one or more network interfaces 808, and one or more computer-readable medium(s) 812 storing software instructions. Each of these components may be coupled by bus 810, and in some embodiments, these components may be distributed among multiple physical locations and coupled by a network 110.

Display device(s) 806 may be any known display technology, including but not limited to display devices using Liquid Crystal Display (LCD) or Light Emitting Diode (LED) technology. Processor(s) 802 may use any known processor technology, including but not limited to graphics processors and multi-core processors. Input device(s) 804 may be any known input device technology, including but not limited to a keyboard (including a virtual keyboard), mouse, track ball, camera, and touch-sensitive pad or display. Bus 810 may be any known internal or external bus technology, including but not limited to ISA, EISA, PCI, PCI Express, USB, Serial ATA or FireWire. Computer-readable medium(s) 812 may be any non-transitory medium that participates in providing instructions to processor(s) 802 for execution, including without limitation, non-volatile storage media (e.g., optical disks, magnetic disks, flash drives, etc.), or volatile media (e.g., SDRAM, ROM, etc.).

Computer-readable medium(s) 812 may include various instructions for implementing an operating system 814 (e.g., Mac OS®, Windows®, Linux). The operating system may be multi-user, multiprocessing, multitasking, multithreading, real-time, and the like. The operating system may perform basic tasks, including but not limited to: recognizing input from input device(s) 804; sending output to display device(s) 806; keeping track of files and directories on computer-readable medium(s) 812; controlling peripheral devices (e.g., disk drives, printers, etc.) which can be controlled directly or through an I/O controller; and managing traffic on bus 810. Network communications instructions 816 may establish and maintain network connections (e.g., software for implementing communication protocols, such as TCP/IP, HTTP, Ethernet, telephony, etc.).

Database processing engine 818 may include instructions that enable computing device 800 to implement one or more methods as described herein. Application(s) 820 may be an application that uses or implements the processes described herein and/or other processes. The processes may also be implemented in operating system 814. For example, application(s) 820 and/or operating system 814 may execute one or more operations to model an epidemic and/or formulate epidemic abate strategies.

The described features may be implemented in one or more computer programs that may be executable on a programmable system including at least one programmable processor coupled to receive data and instructions from, and to transmit data and instructions to a data storage system (e.g., database(s) 108), at least one input device, and at least one output device. A computer program is a set of instructions that can be used, directly or indirectly, in a computer to perform a certain activity or bring about a certain result. A computer program may be written in any form of programming language (e.g., Janusgraph, Gremlin, Sandbox, SQL, Objective-C, Java), including compiled or interpreted languages, and it may be deployed in any form, including as a stand-alone program or as a module, component, subroutine, or other unit suitable for use in a computing environment.

Suitable processors for the execution of a program of instructions may include, by way of example, both general and special purpose microprocessors, and the sole processor or one of multiple processors or cores, of any kind of computer. Generally, a processor may receive instructions and data from a read-only memory or a random-access memory or both. The essential elements of a computer may include a processor for executing instructions and one or more memories for storing instructions and data. Generally, a computer may also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data may include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory may be supplemented by, or incorporated in, ASICs (application-specific integrated circuits).

To provide for interaction with a user, the features may be implemented on a computer having a display device such as an LED or LCD monitor for displaying information to the user and a keyboard and a pointing device such as a mouse or a trackball by which the user can provide input to the computer.

The features may be implemented in a computer system that includes a back-end component, such as a data server, or that includes a middleware component, such as an application server or an Internet server, or that includes a front-end component, such as a client computer having a graphical user interface or an Internet browser, or any combination thereof. The components of the system may be connected by any form or medium of digital data communication such as a communication network. Examples of communication networks include, e.g., a telephone network, a LAN, a WAN, and the computers and networks forming the Internet.

The computer system may include clients and servers. A client and server may generally be remote from each other and may typically interact through a network. The relationship of client and server may arise by virtue of computer programs running on the respective computers and having a client-server relationship to each other.

One or more features or steps of the disclosed embodiments may be implemented using an API. An API may define one or more parameters that are passed between a calling application and other software code (e.g., an operating system, library routine, function) that provides a service, that provides data, or that performs an operation or a computation.

The API may be implemented as one or more calls in program code that send or receive one or more parameters through a parameter list or other structure based on a call convention defined in an API specification document. A parameter may be a constant, a key, a data structure, an object, an object class, a variable, a data type, a pointer, an array, a list, or another call. API calls and parameters may be implemented in any programming language. The programming language may define the vocabulary and calling convention that a programmer will employ to access functions supporting the API.

In some implementations, an API call may report to an application the capabilities of a device running the application, such as input capability, output capability, processing capability, power capability, communications capability, etc.

While various embodiments have been described above, one having skill in the art will appreciate that they have been presented by way of example and not limitation. It will be apparent to persons skilled in the relevant art(s) that various changes in form and detail can be made therein without departing from the spirit and scope. In fact, after reading the above description, it will be apparent to one skilled in the relevant art(s) how to implement alternative embodiments. For example, other steps may be provided, or steps may be eliminated, from the described flows, and other components may be added to, or removed from, the described systems. Accordingly, other implementations are within the scope of the following claims.

In addition, one having skill in the art will appreciate that any figures which highlight the functionality, and advantages are presented for example purposes only. The disclosed methodology and system are each sufficiently flexible and configurable such that they may be utilized in ways other than that shown.

Although the term “at least one” may often be used in the specification, claims and drawings, the terms “a”, “an”, “the”, “said”, etc. also signify “at least one” or “the at least one” in the specification, claims and drawings.

It is the applicant's intent that only claims that include the express language “means for” or “step for” be interpreted under 35 U.S.C. 112(f). Claims that do not expressly include the phrase “means for” or “step for” are not to be interpreted under 35 U.S.C. 112(f).

Although the present invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the scope of the appended claims. For example, it is to be understood that the present invention contemplates that, to the extent possible, one or more features of any embodiment can be combined with one or more features of any other embodiment.

What is claimed is:

1. A system, comprising:
one or more processors, wherein the one or more processors are configured to implement instructions for:
aggregating population data and epidemic data from one or more external databases;
inputting the one or more populations as input for an open Markov model;
identifying a set of predetermined time-dependent states, wherein a set number of the predetermined time-dependent state are associated with epidemic interventions;
identifying an initial state from the set of predetermined time-dependent states for each of the one or more populations;
assigning, for each state of the set of predetermined time-dependent states, a transition probability to the one or more populations;
projecting, by the open Markov model for a specific future point in time, a projected state of the number of time-dependent states, for each of the one or more populations;
generating an intervention trend ratio by comparing the one or more populations at their respective initial state to the one or more populations at their projected state;
receiving the projected state for each of the one or more populations as input for a redress model;
identifying remedies for abating an epidemic in a specific local geographic region, wherein the remedies include one or more resources and respective resource values associated with the specific local geographic region;
simulating, via the redress model, an impact of the resources in abating the epidemic in the specific local geographic region;
determining a final intervention trend ratio;
receiving the one or more resources and respective resource values from the redress model as input for an abatement model;
determining an aggregate value associated with implementing each of the one or more resources in abating the epidemic in the specific local geographic region;
determining a yearly value by applying an inflation value to the aggregate value; and
projecting a yearly value for each year within a predetermined number of future years.

2. The system of claim 1, further comprising wherein, the aggregated population data and epidemic data converting the population data and epidemic data into one or more populations may be converted into a second format for use by the open Markov model.

3. The system of claim 1, wherein assigning the set of predetermined time-dependent states, further comprises wherein the transition probability indicates a likelihood that the one or more populations will transition to a next state in the set of predetermined time-dependent state at a specific point in time.

4. The system of claim 1, wherein determining a final intervention trend ratio, further comprises wherein the final intervention trend ratio is indicative of an efficacy of the resources abating the epidemic in the specific local geographic region.

5. The system of claim 1, further comprising bi-directional long short term memory (LSTM) network configured to generate recommendation metric indicative of a degree to which one or more resources need to be adjusted to align with one or more objectives of an epidemic abatement plan.

6. The system of claim 1, further comprising an evaluation model configured to receive output from the open Markov model, the redress model, abatement model, to generate a progress metric indicative of objective attainment associated with an epidemic abatement plan.

7. The system of claim 1, wherein the one or more processors are further configured to implement instructions for conducting one or more analytical processes including one or more of: a regression analysis, a matching analysis, or a propensity score evaluation, to generate projections regarding the efficacy of an epidemic abatement plan.

8. A computer-implemented method comprising:
aggregating population data and epidemic data from one or more external databases;
inputting the one or more populations as input for an open Markov model;
identifying a set of predetermined time-dependent states, wherein a set number of the predetermined time-dependent state are associated with epidemic interventions;
identifying an initial state from the set of predetermined time-dependent states for each of the one or more populations;
assigning, for each state of the set of predetermined time-dependent states, a transition probability to the one or more populations;
projecting, by the open Markov model for a specific future point in time, a projected state of the number of time-dependent states, for each of the one or more populations;
generating an intervention trend ratio by comparing the one or more populations at their respective initial state to the one or more populations at their projected state;
receiving the projected state for each of the one or more populations as input for a redress model;
receiving the projected state for each of the one or more populations as input for a redress model;
identifying remedies for abating an epidemic in a specific local geographic region, wherein the remedies include one or more resources and respective resource values associated with the specific local geographic region;
simulating, via the redress model, an impact of the resources in abating the epidemic in the specific local geographic region;
determining a final intervention trend ratio;
receiving the one or more resources and respective resource values from the redress model as input for an abatement model;
determining an aggregate value associated with implementing each of the one or more resources in abating the epidemic in the specific local geographic region;
determining a yearly value by applying an inflation value to the aggregate value; and
projecting a yearly value for each year within a predetermined number of future years.

9. The computer-implemented method of claim 8, further comprising wherein, the aggregated population data and epidemic data converting the population data and epidemic data into one or more populations may be converted into a second format for use by the open Markov model.

10. The computer-implemented method of claim 8, wherein assigning the set of predetermined time-dependent states, further comprises wherein the transition probability indicates a likelihood that the one or more populations will transition to a next state in the set of predetermined time-dependent state at a specific point in time.

11. The computer-implemented method of claim 8, wherein determining a final intervention trend ratio, further comprises wherein the final intervention trend ratio is indicative of an efficacy of the resources abating the epidemic in the specific local geographic region.

12. The computer-implemented method of claim 8, further comprising implementing the computer-implemented method on bi-directional long short term memory (LSTM) network configured to generate a recommendation metric indicative of a degree to which one or more resources need to be adjusted to align with one or more objectives of an epidemic abatement plan.

13. The computer-implemented method of claim 8, further comprising an evaluation model configured to receive output from the open Markov model, the redress model, abatement model, to generate a progress metric indicative of objective attainment associated with an epidemic abatement plan.

14. The computer-implemented method of claim 8, further comprises conducting one or more analytical processes including one or more of: a regression analysis, a matching analysis, or a propensity score evaluation, to generate projections regarding the efficacy of an epidemic abatement plan.

15. A non-transitory computer-readable medium storing instructions that when executed by one or more processors, cause the one or more processors to implement a computer-implemented method for:
aggregating population data and epidemic data from one or more external databases;
inputting the one or more populations as input for an open Markov model;
identifying a set of predetermined time-dependent states, wherein a set number of the predetermined time-dependent state are associated with epidemic interventions;
identifying an initial state from the set of predetermined time-dependent states for each of the one or more populations;
assigning, for each state of the set of predetermined time-dependent states, a transition probability to the one or more populations;
projecting, by the open Markov model for a specific future point in time, a projected state of the number of time-dependent states, for each of the one or more populations;
generating an intervention trend ratio by comparing the one or more populations at their respective initial state to the one or more populations at their projected state;
receiving the projected state for each of the one or more populations as input for a redress model;
receiving the projected state for each of the one or more populations as input for a redress model;
identifying remedies for abating an epidemic in a specific local geographic region, wherein the remedies include one or more resources and respective resource values associated with the specific local geographic region;
simulating, via the redress model, an impact of the resources in abating the epidemic in the specific local geographic region;
determining a final intervention trend ratio;
receiving the one or more resources and respective resource values from the redress model as input for an abatement model;
determining an aggregate value associated with implementing each of the one or more resources in abating the epidemic in the specific local geographic region;
determining a yearly value by applying an inflation value to the aggregate value; and
projecting a yearly value for each year within a predetermined number of future years.

16. The non-transitory computer-readable medium of claim 15, further storing instructions for aggregating population data and epidemic data converting the population data and epidemic data into one or more populations may be converted into a second format for use by the open Markov model.

17. The non-transitory computer-readable medium of claim 15, further storing instructions for assigning the set of predetermined time-dependent states, further comprises wherein the transition probability indicates a likelihood that the one or more populations will transition to a next state in the set of predetermined time-dependent state at a specific point in time.

18. The non-transitory computer-readable medium of claim 15, further storing instructions for determining a final intervention trend ratio, further comprises wherein the final intervention trend ratio is indicative of an efficacy of the resources abating the epidemic in the specific local geographic region.

19. The non-transitory computer-readable medium of claim 15, further storing instructions for implementing the computer-implemented method on bi-directional long short term memory (LSTM) network configured to generate a recommendation metric indicative of a degree to which one or more resources need to be adjusted to align with one or more objectives of an epidemic abatement plan.

20. The non-transitory computer-readable medium of claim 15, further storing instructions for conducting one or more analytical processes including one or more of: a regression analysis, a matching analysis, or a propensity score evaluation, to generate projections regarding the efficacy of an epidemic abatement plan.
